(19) United States
(12) Patent Application Publication
(10) Pub. No.: US 2026/0182924 A1
(43) Pub. Date: Jul. 2, 2026

(54) Physiological Measurement Initiation Using Multiple Sensors

(71) Applicant: iRhythm Technologies, Inc., San Francisco, CA (US)

(72) Inventor: Monica C. Lin, San Francisco, CA (US)

(73) Assignee: iRhythm Technologies, Inc., San Francisco, CA (US)

(21) Appl. No.: 19/436,849

(22) Filed: Dec. 30, 2025

Related U.S. Application Data
(60) Provisional application No. 63/740,289, filed on Dec. 30, 2024.

(51) Int. Cl. A61B 5/00; A61B 5/318

(52) U.S. Cl. CPC A61B 5/7214; A61B 5/318; A61B 5/4809; A61B 5/6823; A61B 5/6824; A61B 2562/0219

(57) ABSTRACT

Techniques for physiological measurement initiation using multiple sensors are described. In one or more implementations, first physiological data are collected from a first sensor that measures a first physiological feature of an individual. The first physiological data are analyzed in real-time to detect a physiological condition of the individual. A second sensor is activated, responsive to detection of the physiological condition, to begin collecting second physiological data measuring a second physiological feature of the individual.

### RELATED APPLICATIONS

This application claims priority to U.S. Provisional Application No. 63/740,289, filed December 30, 2024, and titled “Physiological Measurement Initiation Using Multiple Sensors,” which is hereby incorporated by reference in its entirety.

### BACKGROUND

Wearable health monitoring devices have become increasingly prevalent, often incorporating multiple sensors to measure various physiological parameters such as heart activity, movement, and blood oxygen levels. These devices typically collect data continuously or at predetermined intervals, which can lead to significant power consumption and data storage requirements. While continuous monitoring provides comprehensive health information, it is often unnecessary and computationally inefficient, such as when physiological measurements are relevant under specific conditions or during particular activities.

### BRIEF DESCRIPTION OF THE DRAWINGS

FIG. 1 is a block diagram of a non-limiting example of an environment that is operable to employ techniques for physiological measurement initiation using multiple sensors as described herein.

FIG. 2 depicts a non-limiting example of a monitoring device.

FIGS. 3A and 3B illustrate an example showing an interaction between the monitoring device and the auxiliary device.

FIG. 4 illustrates a first example of physiological measurement initiation using multiple sensors based on an ECG signal during awake and asleep states.

FIG. 5 depicts a second example illustrating physiological measurement initiation using multiple sensors based on an accelerometer signal during awake and asleep states.

FIG. 6 illustrates a method for physiological measurement initiation using multiple sensors.

### DETAILED DESCRIPTION

Conventional physiological monitoring systems often employ continuous data collection from multiple sensors, leading to significant power consumption and data storage demands. However, these systems frequently struggle with efficient resource utilization when collecting data from multiple sensor types. This approach, while comprehensive, is inefficient when certain measurements are relevant under specific conditions and/or during particular activities and not relevant under other conditions and/or activities. For example, conventional devices may continuously collect both electrocardiogram (ECG) and photoplethysmogram (PPG) data, even when one type of data may not be relevant, which is computationally expensive and leads to slow and/or degraded performance of the device. By way of example, the continuous operation of multiple sensor types regardless of a physiological relevance of the data being collected may lead to increased battery drain and excessive data storage.

Accordingly, techniques for physiological measurement initiation using multiple sensors are described. In an example, first physiological data is collected from a first sensor that measures a first physiological feature, such as ECG data and/or accelerometer data. The first physiological data is analyzed in real-time to detect a specific physiological condition, such as one or more user behaviors, states, and/or actions (e.g., sleep states, activity levels, and/or other physiological states). When the physiological condition is detected, a second sensor is activated to begin collecting second physiological data measuring a second physiological feature, such as PPG data.

For instance, first physiological data from a chest-worn monitoring device may be continuously analyzed to determine that an individual is asleep. By way of example, the first physiological data may include ECG data, and a sleep state may be detected (e.g., by an activation algorithm) based on various indicators like slowed respiration rate, heart rate, and/or a stabilization of cardiac intervals over multiple consecutive heartbeats. Alternatively, or in addition, the first physiological data may include accelerometer data, which may be analyzed (e.g., by the activation algorithm) to detect when movement levels indicate the sleep state, such as by remaining within a defined range for a threshold duration. Upon detecting the individual is asleep, a second sensor may be activated to collect additional data relevant to sleep analysis. The second sensor may be in the chest-worn monitoring device or in a separate (e.g., auxiliary) device that is worn in a different body location (e.g., an extremity). By way of example, the second sensor may be a PPG sensor in a wrist-worn auxiliary device. Accordingly, the selective activation may be implemented within a single wearable monitoring device or through a combination of a primary monitoring device (e.g., the chest-worn monitoring device) and the auxiliary device worn on an extremity, such as a wrist, finger, or ankle. Accordingly, the activation algorithm may activate the second sensor based on the first physiological data collected by the first sensor, such as to selectively collect the second physiological data via communication of an activation signal to the second sensor.

A system implementing the physiological measurement initiation techniques described herein may further detect (e.g., via the activation algorithm) when the physiological condition has ceased based on either the first physiological data and/or the second physiological data and subsequently terminate data collection by the second sensor. This dynamic approach to sensor activation and deactivation, which may include powering on and off the second sensor and/or adjusting a power level of the second sensor, may significantly improve energy efficiency and data management.

By selectively activating sensors based on real-time physiological conditions, the techniques described herein offer several advantages over conventional systems. By way of example, the techniques described herein may reduce overall power consumption, extend battery life, and/or support extended continuous monitoring periods. Additionally, the techniques described herein may optimize data storage by collecting specific types of data when relevant, thus reducing a volume of data to be processed and analyzed. This may further result in increased computational efficiency and enable more focused data analysis. By way of example, the targeted data collection may enhance a quality and relevance of collected information, which improves accuracy of subsequent analyses and diagnoses.

Moreover, in some applications, such as combined ambulatory cardiac monitoring and home sleep testing, the techniques described herein may enable continuous ECG collection for cardiac assessment while activating PPG collection selectively during sleep periods, which may provide comprehensive health information while optimizing power and storage utilization. It is to be appreciated that the flexibility in sensor activation methods and device configurations described herein enable the techniques to be adapted to a range of monitoring scenarios and clinical applications, enhancing practical applicability in various healthcare settings.

The techniques described herein provide a technical solution for selective sensor activation based on physiological condition detection in the field of physiological monitoring. The techniques described herein utilize wearable devices including a monitoring device and an auxiliary device that measure physiological parameters from a body of an individual. These devices include sensors such as electrodes for ECG measurement, optical sensors for PPG measurement, accelerometers, and respiration sensors that detect and convert physiological phenomena into electrical signals. The monitoring device and the auxiliary device generate time-stamped physiological data that is transmitted wirelessly or via wired connections to an analysis platform for processing.

In one or more implementations, an analysis platform performs real-time processing of the physiological data, as the data is received from the monitoring device and/or the auxiliary device. As used herein, the term “real-time” may refer to the processing or analysis of data as the data is received and/or generated, without intentional delay between data collection and processing. Real-time processing, for instance, may enable an immediate or near-immediate response to detected conditions, such as generating activation signals within seconds or sub-seconds of detecting physiological conditions. Real-time processing may include processing data in streaming fashion as the data arrives, rather than waiting for data collection to be completed before beginning analysis. By way of example, the activation algorithm may analyze incoming physiological data streams in real-time during an ongoing observation period to detect a physiological condition and generate an activation signal in response to the detected physiological condition. This real-time processing enables the analysis platform to continuously or semi-continuously (e.g., at a predetermined frequency) monitor a physiological state of an individual and control sensor operation in response to a change in the physiological state. By way of example, the analysis platform may receive ECG data in real-time, evaluate parameters such as heart rate and/or respiration rate as the ECG data arrives, and generate an activation signal on-the-fly to trigger PPG data collection.

As used herein, the term “continuous monitoring” may refer to an ongoing collection of physiological data over an extended period without predetermined interruptions. Continuous monitoring, for example, may include collecting data at regular intervals throughout a monitoring period, which may last hours, days, or weeks. Continuous monitoring may enable a detection of physiological conditions and patterns that occur at unpredictable times during the monitoring period.

The techniques described herein may adjust how the monitoring device and/or the auxiliary device operate. By way of example, the activation signal generated by the analysis platform may cause activation of a sensor (e.g., powering on of a component) to begin data collection by the sensor. In at least one implementation, an absence of the activation signal, or a transmission of a deactivation signal, may cause deactivation of the sensor (e.g., powering down of the component) to cease data collection by the sensor. This represents a change to the operation of a corresponding device that improves its technical functioning by enabling selective data collection based on physiological relevance. The selectively collected physiological data generated through this process provides a technical improvement over conventional multi-device monitoring systems that continuously collect sensor data from multiple sensors, enabling more efficient power utilization and storage management. Accordingly, the techniques described herein improve the functioning of multi-device monitoring systems.

In some aspects, the techniques described herein relate to a method for physiological measurement initiation, including: collecting first physiological data from a first sensor measuring a first physiological feature of an individual; analyzing the first physiological data in real-time to detect a physiological condition of the individual; and responsive to detecting the physiological condition, activating a second sensor to collect second physiological data measuring a second physiological feature of the individual.

In some aspects, the techniques described herein relate to a method, further including: detecting that the physiological condition has ceased based on one or more of the first physiological data or the second physiological data; and terminating collection of the second physiological data by the second sensor responsive to the detecting.

In some aspects, the techniques described herein relate to a method, wherein terminating collection of the second physiological data by the second sensor responsive to the detecting includes transmitting a deactivation signal to the second sensor responsive to the detecting.

In some aspects, the techniques described herein relate to a method, wherein the activating the second sensor includes powering on the second sensor.

In some aspects, the techniques described herein relate to a method, wherein the first sensor is included in a wearable monitoring device attached to a chest region of the individual, and the second sensor is included in an auxiliary device attached to an extremity of the individual.

In some aspects, the techniques described herein relate to a method, wherein the first sensor and the second sensor are included in a single wearable monitoring device.

In some aspects, the techniques described herein relate to a method, wherein the first physiological data include one or more of electrocardiography (ECG) data or accelerometer data, and the second physiological data include photoplethysmogram (PPG) data.

In some aspects, the techniques described herein relate to a method, wherein the physiological condition is an asleep condition of the individual.

In some aspects, the techniques described herein relate to a method, wherein the first physiological data include ECG data, and analyzing the first physiological data to detect the physiological condition of the individual includes: detecting, as the first physiological feature, a cardiac feature in the ECG data; and detecting the physiological condition based on the cardiac feature.

In some aspects, the techniques described herein relate to a method, wherein the first physiological data include accelerometer data, and analyzing the first physiological data to detect the physiological condition of the individual includes: detecting, as the first physiological feature, a movement feature in the accelerometer data; and detecting the physiological condition based on the movement feature.

In some aspects, the techniques described herein relate to a system for physiological measurement initiation, including: a first sensor configured to collect first physiological data measuring a first physiological feature of an individual; a second sensor configured to collect second physiological data measuring a second physiological feature of the individual; and a processing device configured to: analyze the first physiological data in real-time to detect a physiological condition of the individual; and activate the second sensor to collect the second physiological data responsive to detecting the physiological condition.

In some aspects, the techniques described herein relate to a system, wherein the processing device is further configured to: detect that the physiological condition has ceased based on one or more of the first physiological data or the second physiological data; and in response to detecting that the physiological condition has ceased, terminate collection of the second physiological data by the second sensor.

In some aspects, the techniques described herein relate to a system, wherein the processing device is further configured to: terminate collection of the second physiological data by transmitting a deactivation signal to the second sensor after a predetermined time duration or after a predetermined number of measurements has been collected by the second sensor.

In some aspects, the techniques described herein relate to a system, wherein the processing device is configured to activate the second sensor by transmitting an activation signal to the second sensor.

In some aspects, the techniques described herein relate to a system, wherein the first sensor is included in a wearable monitoring device configured to be attached to a chest region of the individual, and the second sensor is included in an auxiliary device configured to be attached to an extremity of the individual.

In some aspects, the techniques described herein relate to a system, wherein the first sensor and the second sensor are included in a single wearable monitoring device.

In some aspects, the techniques described herein relate to a system, wherein the first sensor is one or more of an electrocardiography (ECG) sensor or an accelerometer sensor, and the second sensor is a photoplethysmogram (PPG) sensor.

In some aspects, the techniques described herein relate to a method for physiological measurement initiation, including: continuously collecting first physiological data from a first sensor that measures a first physiological feature of an individual over an observation period; analyzing the first physiological data in real-time to detect a physiological condition of the individual at a first time point during the observation period; responsive to detecting the physiological condition, activating a second sensor at the first time point to collect second physiological data measuring a second physiological feature of the individual; at a second time point during the observation period, detecting cessation of the physiological condition based on one or more of the first physiological data or the second physiological data; and deactivating the second sensor at the second time point responsive to detecting the cessation of the physiological condition.

In some aspects, the techniques described herein relate to a method, wherein the first physiological data include one or more of ECG data or accelerometer data, the second physiological data include PPG data, and the physiological condition is an asleep condition of the individual.

In some aspects, the techniques described herein relate to a method, wherein the first sensor is included in a wearable monitoring device attached to a chest region of the individual, and the second sensor is included in an auxiliary device attached to an extremity of the individual.

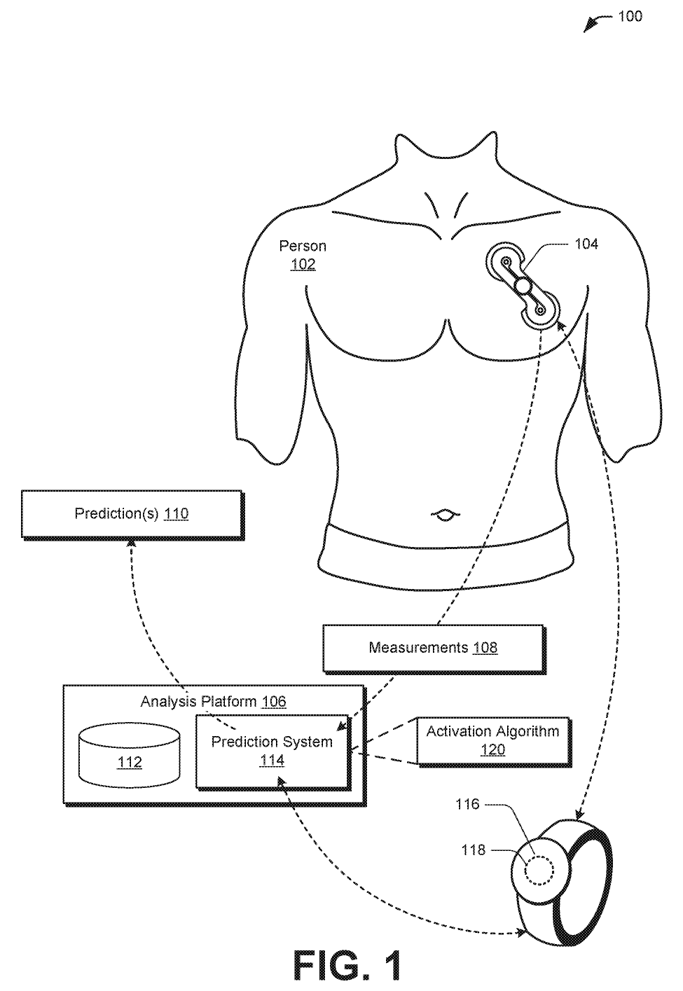

FIG. 1 is a block diagram of a non-limiting example 100 of an environment that is operable to employ techniques for physiological measurement initiation using multiple sensors as described herein. The illustrated example 100 includes a monitored subject, e.g., a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. In some scenarios, for instance, the monitoring device 104 may be provided to record electrical activity of a heart of the person 102 over an observation period, e.g., lasting some number of seconds or minutes, lasting multiple days, and so on. By way of example, the person 102 may have a magnitude of electrical potential of a heart monitored over time to produce one or more electrocardiograms, which may be used to determine or predict any of a variety of events. Alternatively, or in addition, the monitoring device 104 may be used to output the measurements 108 (e.g., a time sequence of measurements such as a time sequence of electric potential measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing the monitoring device 104. In one or more implementations, the instructions may be provided as part of a kit, e.g., written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

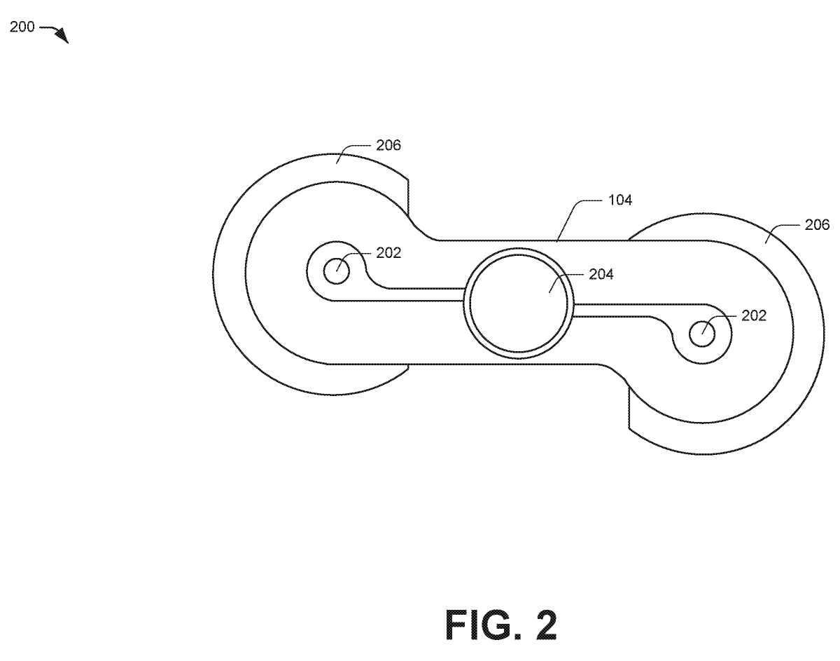

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, a temperature sensor, a heat flux sensor, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements.

In one or more implementations, the monitoring device 104 may be configured to offload measurements and/or other data from the monitoring device during the course of the observation period. By way of example, the monitoring device 104 may offload the measurements by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from storage of the device.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate one or more predictions 110. For example, the measurements 108 (and/or other measurements such as accelerometer data and oxygen saturation (SpO2) measurements) may be processed by a smartphone associated with a user, a smartphone or other dedicated device associated with the monitoring device 104, and/or one or more server computers at a data center or other location that can be utilized by an entity associated with the monitoring device 104, to name just a few. In other words, those other devices may implement at least a portion of the prediction system 114.

In one or more implementations, the monitoring device 104 is configured to transmit the measurements 108 to an external device over a wired connection with the external device, e.g., via USB-C or some other physical, communicative coupling. As used herein, an “external device” is meant to denote a device that is not body-worn. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with additional (e.g., separate) devices using one or more wireless communication protocols or techniques. By way of example, the monitoring device 104 may communicate with the additional devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc. As noted above, examples of such additional measurements include but are not limited to accelerometer data and/or oxygen saturation (e.g., SpO2) measurements.

In one or more implementations, the analysis platform 106 may be implemented in whole or in part at the monitoring device 104. Alternatively, or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform 106 includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that in variations, the prediction system 114 may output different combinations of multiple predictions.

In at least one implementation, the prediction system 114 uses machine learning and/or one or more algorithms to generate the one or more predictions 110. The one or more predictions 110 may include determinations regarding physiological conditions, health states, and/or events detected from the physiological data. In one or more implementations, the one or more predictions 110 may include an assessment or diagnosis related to a physiological condition or disease state. By way of example, the one or more predictions 110 may include predictions related to sleep apnea, cardiac arrhythmias, sleep stages, activity levels, and/or other physiological conditions. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the obtained measurements, such as to extract various cardiovascular and/or other features from the sequences of measurements. The illustrated example 100 also includes one or more predictions 110, which corresponds to the output of the prediction system 114.

The illustrated example 100 further includes an auxiliary device 116. In various examples, the auxiliary device 116 is configured as a wearable device that complements the monitoring device 104 in collection of physiological data from the person 102. In some aspects, the auxiliary device 116 may be designed to be worn on a different part of the body than the monitoring device 104, such as on a wrist, finger, ankle, or the like. The auxiliary device 116 may incorporate various sensors to capture different types of physiological measurements, which may include, but are not limited to, heart rate, blood oxygen saturation, skin temperature, motion data, PPG data, and so forth. In the illustrated example 100, the one or more sensors 118 are shown as a dashed outline to indicate that the one or more sensors 118 may be positioned on a back surface of the auxiliary device 116. In implementations where the auxiliary device 116 is configured as a wrist-worn device, for example, the one or more sensors 118 may be disposed on a skin-contacting surface that faces inward toward the wrist when worn. The positioning of the one or more sensors 118 on the back surface may enable the sensors to detect physiological signals through contact with or proximity to skin, such as optical signals for photoplethysmography (PPG) measurements, electrical signals, temperature measurements, and/or other physiological parameters. This is by way of example and not limitation, and a variety of properties and form factors of the auxiliary device 116 are considered.

For instance, the auxiliary device 116 may share one or more properties with the monitoring device 104 as described above and below in more detail. The auxiliary device 116, for instance, is configured to communicate with the analysis platform 106 to perform a variety of functionality. In one or more examples, the auxiliary device 116 is configured to offload measurements during the observation period, such as through wired or wireless connections to an external computing device. The auxiliary device 116 may also employ data compression techniques to reduce battery usage and facilitate extended wear. Additionally, the auxiliary device 116 may be designed with various communication capabilities, allowing it to transmit data directly to the analysis platform 106 or to other devices such as smartphones or tablets.

In various examples, the auxiliary device 116 is configured to communicate with the analysis platform 106 and/or the monitoring device 104, such as to receive one or more signals to influence processing and/or data collection operations of the auxiliary device 116. For instance, as further described in more detail below in various examples, the monitoring device 104 includes a first sensor, and the auxiliary device 116 includes a second sensor. Based on readings detected from the first sensor, the auxiliary device 116 initiates various functionality of the second sensor, e.g., activation. The activation of the second sensor may be responsive to detection of a physiological condition based on a real-time analysis of data collected by the first sensor, enabling selective data collection that optimizes power consumption and storage utilization. This is by way of example and not limitation, and in various examples, the first sensor and the second sensor are integrated into a single device, e.g., the auxiliary device 116 and/or the monitoring device 104. It should be understood that the operations and functionality described herein as performed by the auxiliary device 116 are likewise performable by the monitoring device 104, and the operations and functionality described herein as performed by the monitoring device 104 are likewise performable by the auxiliary device 116.

In the illustrated example 100, the analysis platform 106 includes an activation algorithm 120. The activation algorithm 120 represents functionality of the prediction system 114 to analyze the measurements 108 from the monitoring device 104 in real-time and determine when to activate the one or more sensors 118 of the auxiliary device 116 to begin collecting additional physiological data. In one or more implementations, the activation algorithm 120 may analyze ECG data and/or accelerometer data from the monitoring device 104 to detect a physiological condition, such as determining when the person 102 is asleep. The activation algorithm 120 may look for indicators such as slowed respiration rate, reduced heart rate, decreased movement, and/or other metrics indicative of sleep or other physiological states. Upon detecting the physiological condition, the activation algorithm 120 may generate an activation signal to trigger the one or more sensors 118 of the auxiliary device 116 (and/or one or more other sensors of the monitoring device 104) to begin data collection, such as by activating a PPG sensor to collect PPG data.

By selectively activating sensors based on detected physiological conditions, the activation algorithm 120 enables significant power savings and storage optimization. For example, in a combined ambulatory cardiac monitor and home sleep test implementation, the monitoring device 104 may continuously measure ECG and/or accelerometer data, which may consume less power than optical sensors typically used for PPG. The activation algorithm 120 may selectively activate PPG sensing capability when the person 102 is asleep, thereby saving both storage space and battery power by avoiding PPG data collection during waking hours, which may be less physiologically relevant. The activation algorithm 120 may also detect when the physiological condition has ceased and generate a deactivation signal to terminate the PPG sensing capability, further optimizing resource utilization. In one or more implementations, the activation algorithm 120 may receive additional inputs (e.g., in addition to the measurements 108), such as inputs defining typical sleep/wake times, an age of the person 102, a height of the person 102, a weight of the person 102, and so forth. At least a portion of these additional inputs may be received from a user. In some examples, at least a portion of these inputs may be used to define threshold(s) for detecting the physiological condition. Alternatively, or in addition, the user may adjust the threshold(s).

Alternatively, or in addition, the activation algorithm 120 may utilize wireless communication methods to transmit activation signals between the monitoring device 104 and the auxiliary device 116, and/or through a gateway device that is not body-worn. In one or more implementations, the activation algorithm 120 may be implemented on-device within the monitoring device 104 and/or the auxiliary device 116 to enable real-time analysis and sensor control. In other scenarios, the activation algorithm 120 may be implemented in a cloud-based system, with the monitoring device 104 and the auxiliary device 116 being cloud-connected directly to the analysis platform 106 or connecting to the analysis platform 106 through a gateway device such as a smartphone, tablet, or another type of dedicated gateway.

Accordingly, it is to be appreciated that although the analysis platform 106 is shown separately from the monitoring device 104 and the auxiliary device 116, in one or more implementations, the analysis platform 106 may be implemented in whole or in part at the monitoring device 104. Alternatively, or in addition, the analysis platform 106 may be implemented in whole or in part at the auxiliary device 116. In other implementations, the analysis platform 106, or portions thereof (e.g., the activation algorithm 120) may be implemented using one or more computing devices external to the monitoring device 104 and the auxiliary device 116, such as one or more computing devices associated with the person 102 (e.g., a mobile phone, tablet device, laptop, desktop, or smartwatch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices. The activation algorithm 120 may similarly be implemented in whole or in part at the monitoring device 104, the auxiliary device 116, or a separate device. The analysis platform 106, the monitoring device 104, or the auxiliary device 116, for instance, may serve as a processing device for executing the activation algorithm 120 based on computer-readable instructions stored in a non-transitory memory.

In this way, the illustrated example 100 enables selective sensor activation based on a real-time analysis of physiological data. The analysis platform 106, through the activation algorithm 120, may coordinate data collection from the monitoring device 104 and/or the auxiliary device 116 to achieve efficient physiological monitoring while reducing power consumption and efficiently storing data. This approach may provide flexibility in device configurations, as the activation algorithm 120 may be implemented at the monitoring device 104, the auxiliary device 116, or a separate device such as a smartphone or gateway. By enabling selective activation of one or more sensors based on detected physiological conditions, the illustrated example 100 may support comprehensive physiological monitoring with improved energy efficiency and enable extended monitoring periods, particularly for applications such as combined ambulatory cardiac monitoring and home sleep testing where certain measurements are indicated under specific physiological conditions.

FIG. 2 depicts a non-limiting example 200 of a monitoring device. The illustrated example 200 depicts the monitoring device 104. As described above, in various examples the auxiliary device 116 may include one or more properties and/or features of the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of a heart of the person 102, e.g., to produce an electrocardiogram (ECG or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on communications with the one or more sensors 202 that are indicative of some aspect of the person 102, such as the electrical activity of the heart of the person 102. In one or more implementations, the processor further generates one or more communicable packages of data that include one or more of the measurements 108 and/or other measurements, such as accelerometer data and oxygen saturation (SpO2) measurements. Alternatively, or additionally, the processor produces and/or causes storage of other data, which may be used for generating predictions, such as predictions related to sleep apnea or cardiac arrhythmias.

In implementations where the monitoring device 104 is configured for wireless transmission, the transmitter 204 may transmit the measurements wirelessly as a stream of data to a computing device (e.g., the analysis platform 106). In one or more implementations, for instance, the monitoring device 104 is configured to transfer (e.g., transmit and/or receive) information (e.g., electrical potential measurements) via a Bluetooth® Low Energy (BLE) connection. Alternatively, or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

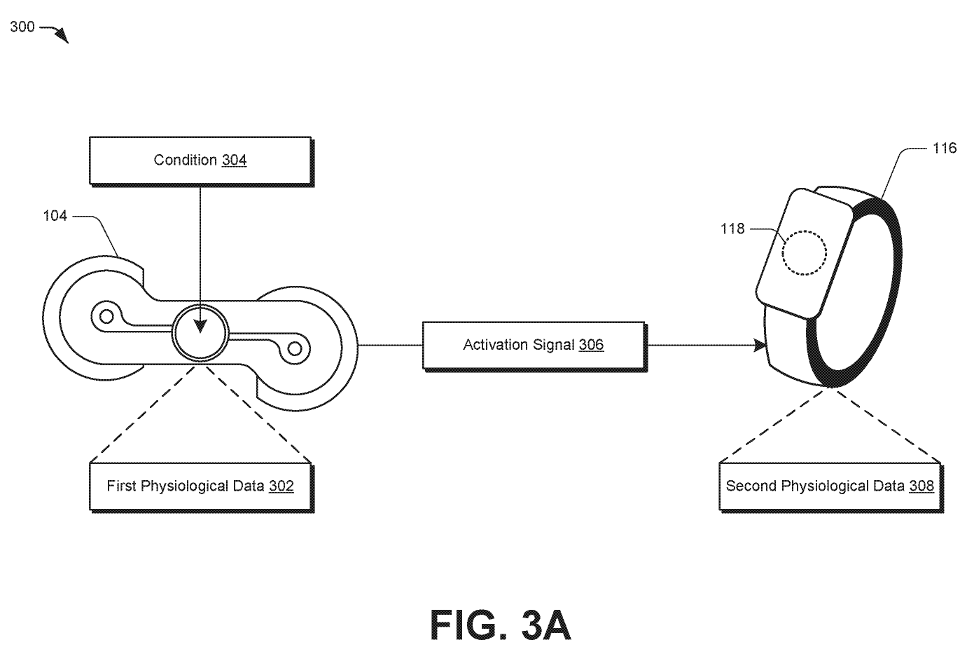

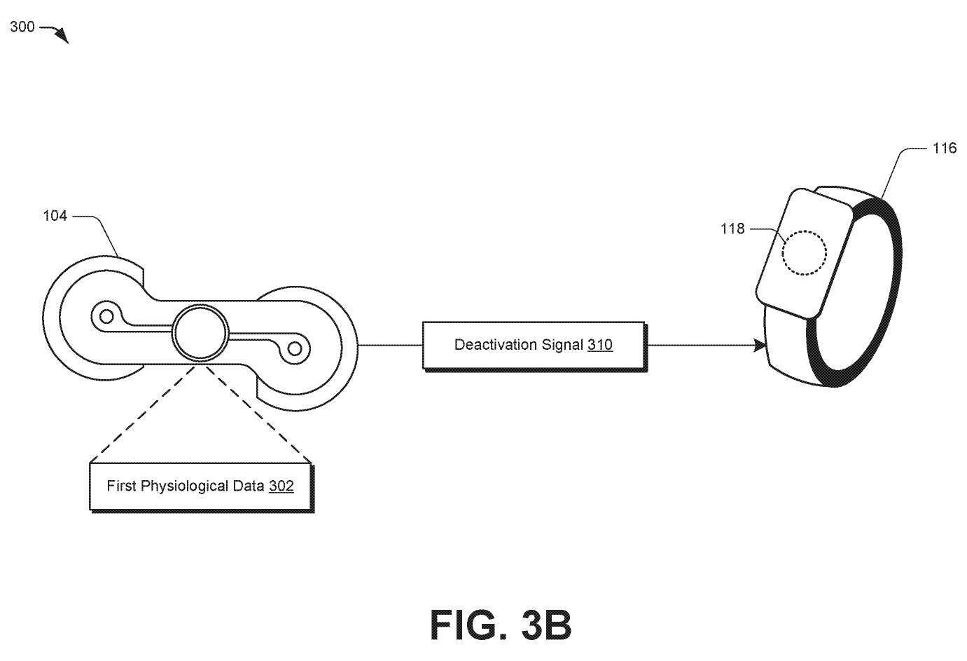

FIGS. 3A and 3B illustrate an example 300 showing an interaction between the monitoring device 104 and the auxiliary device 116. In this example, the monitoring device 104 includes a first sensor that collects first physiological data 302 measuring a first physiological feature. The first physiological feature may include a variety of physiological data. In one example, the first physiological feature includes one or more of an electrocardiography (ECG)-based feature (e.g., a cardiac feature) and/or an accelerometer-based feature (e.g., a movement feature). In some cases, the first physiological data 302 may include derived insights, e.g., a respiration rate that is derived from ECG data.

In the example 300, the monitoring device 104 is implemented as a wearable device attached to a chest region of the person 102. In some cases, the monitoring device 104 may be configured as a combined ambulatory cardiac monitor and home sleep test device. In various examples, the auxiliary device 116 is attached to an extremity of the person 102, such as a wrist or finger. This is by way of example and not limitation and a variety of suitable form factors are considered.

Referring first to FIG. 3A, the first physiological data 302 may be analyzed in real-time (e.g., by the activation algorithm 120) to detect a physiological condition 304. In some cases, the physiological condition 304 may include a determination that, based on analysis of the first physiological data 302, the person 102 is asleep. A variety of other physiological conditions are considered including but not limited to changes in heart rate variability, detection of arrhythmias, changes (e.g., a decrease) in respiration rate, detection of movement patterns indicative of restlessness or periodic limb movements, changes in skin temperature or conductance, and so forth. In some cases, the physiological condition 304 may include a combination of multiple factors analyzed together to determine an overall physiological state. Accordingly, examples of the physiological condition 304 include, but are not limited to, sleep/wake, activity level and/or exercise type, respiratory conditions, arrhythmias, and body position.

The analysis of the first physiological data 302 may be performed by the monitoring device 104 itself, the auxiliary device 116, and/or the analysis platform 106, such as through implementation of one or more analysis algorithms. In one or more implementations, the analysis platform 106 may be implemented on-device within the monitoring device 104 and/or the auxiliary device 116. In other scenarios, the analysis platform 106 may be implemented in a cloud-based system. The monitoring device 104 and the auxiliary device 116 may be cloud-connected directly or may connect to the analysis platform 106 through a gateway device.

When the physiological condition 304 is detected, the monitoring device 104 may generate an activation signal 306. The activation signal 306 is transmitted to the auxiliary device 116. Upon receiving the activation signal 306, the auxiliary device 116 is configured to activate a second sensor (e.g., the one or more sensors 118) to begin collecting second physiological data 308. The second physiological data 308 may measure a second physiological feature. In some cases, the second physiological data may include photoplethysmogram (PPG) data. A variety of second physiological features are considered, including SpO2, pulse rate, respiration rate, and/or another parameter determined or derived from the PPG data. In some implementations, the activation signal 306 is transmitted to initiate activation of the second sensor and is not maintained throughout a measurement period for acquiring the second physiological data 308. In other implementations, the activation signal 306 is transmitted throughout the measurement period (e.g., while the physiological condition 304 is present).

In one or more implementations, the activation signal 306 may further define conditions for operating the second sensor, such as a sampling rate, a drive current, and/or other sensor settings (e.g., an integration time or bias). By way of example, the activation signal 306 may instruct the second sensor to turn on with high current and/or high ambient noise cancellation and/or filtering when ambient light is high when the physiological condition 304 is detected. Alternatively, or in addition, the activation signal 306 may adjust the sampling rate for the second sensor based on an activity level indicated by the physiological condition 304. For instance, a higher activity level may result in a higher sampling rate.

The activation of the second sensor in the auxiliary device 116 may be responsive to the detection of the physiological condition 304. This selective activation approach may allow for efficient use of power and storage resources, as the second sensor may be prevented from collecting data when particular conditions are not met.

Referring now to FIG. 3B, the first physiological data 302 may be analyzed in real-time (e.g., by the activation algorithm 120) to detect that the physiological condition 304 has ceased, which is represented in FIG. 3B by the absence of the physiological condition 304. The analysis may evaluate whether physiological signals in the first physiological data 302 exhibit patterns and/or features (e.g., one or more cardiac features, one or more movement features, or a combination thereof) indicative of a change in state or condition of the person 102, such as a transition from sleep to wakefulness. The analysis may look for indicators such as increased respiration rate, increased heart rate, increased movement, and/or other metrics indicative of wakefulness or other physiological states that differ from the physiological condition 304.

When the analysis detects that the physiological condition 304 has ceased, the monitoring device 104 may generate a deactivation signal 310. The generation of the deactivation signal 310 may occur when cessation criteria are satisfied, such as when signal patterns and/or features indicate a change in the physiological state over a configured number of measurements and/or when signal levels exceed or fall below defined bounds for a threshold duration. In FIG. 3B, the deactivation signal 310 is transmitted to the auxiliary device 116. Upon receiving the deactivation signal 310, the auxiliary device 116 is configured to deactivate the second sensor (e.g., the one or more sensors 118) to cease collecting the second physiological data 308. The deactivation of the second sensor in the auxiliary device 116 may be responsive to the detection that the physiological condition 304 is no longer present. This selective deactivation approach may further optimize power and storage resources by terminating data collection when the physiological condition 304 is no longer present.

In at least one variation, however, the second sensor may be deactivated after a predetermined time duration, a predetermined number of measurements, or upon satisfaction of other predetermined criteria, in addition to or as an alternative to in response to the deactivation signal 310. By way of example, the auxiliary device 116 may be configured to collect the second physiological data 308 for a fixed duration (e.g., a number of seconds, a number of minutes, or a number of hours) following activation by the activation signal 306, after which the second sensor automatically deactivates. Alternatively, or in addition, the auxiliary device 116 may be configured to collect a predetermined number of measurements or data samples after receiving the activation signal 306 before automatically deactivating the second sensor. In some cases, the predetermined criteria may be based on data quality metrics, storage capacity thresholds, battery level thresholds, and/or other operational parameters of the auxiliary device 116. As another example, the predetermined criteria may be based on a clinical application for collecting the second physiological data 308.

In yet another variation, alternatively or in addition, the second sensor (e.g., the one or more sensors 118) may be deactivated based on the second physiological data 308 itself. By way of example, the activation algorithm 120 may analyze the second physiological data 308 to detect patterns and/or features indicative of cessation of the physiological condition 304, such as changes in PPG waveform characteristics, pulse rate patterns, and/or oxygen saturation levels that indicate a transition from sleep to wakefulness. Alternatively, or in addition, the patterns and/or features may include motion artifacts detected based on an accelerometer signal. Upon detecting such patterns and/or features in the second physiological data 308, the activation algorithm 120 may generate the deactivation signal 310. For instance, motion artifacts may indicate that the second physiological data 308 would be unusable due to noise if continued to be collected.

In one or more other variations, the deactivation signal 310 is not generated. Instead, the second sensor may be deactivated in response to the activation signal 306 no longer being received, such as in implementations where the activation signal 306 is transmitted for an entirety of the measurement period for collecting the second physiological data 308.

By utilizing the interaction between the monitoring device 104 and the auxiliary device 116 shown in FIGS. 3A and 3B, the example 300 provides comprehensive physiological monitoring while optimizing power consumption and data collection. The combination of different sensor types and strategic activation and deactivation based on detected conditions enable effective health monitoring, particularly for applications such as combined ambulatory cardiac monitoring and home sleep testing where certain measurements are relevant under specific conditions. This selective sensor activation and deactivation approach may extend battery life, support extended continuous monitoring periods, and optimize data storage by collecting specific types of data when relevant, thereby reducing the volume of data to be processed and analyzed while enhancing the quality and relevance of collected information.

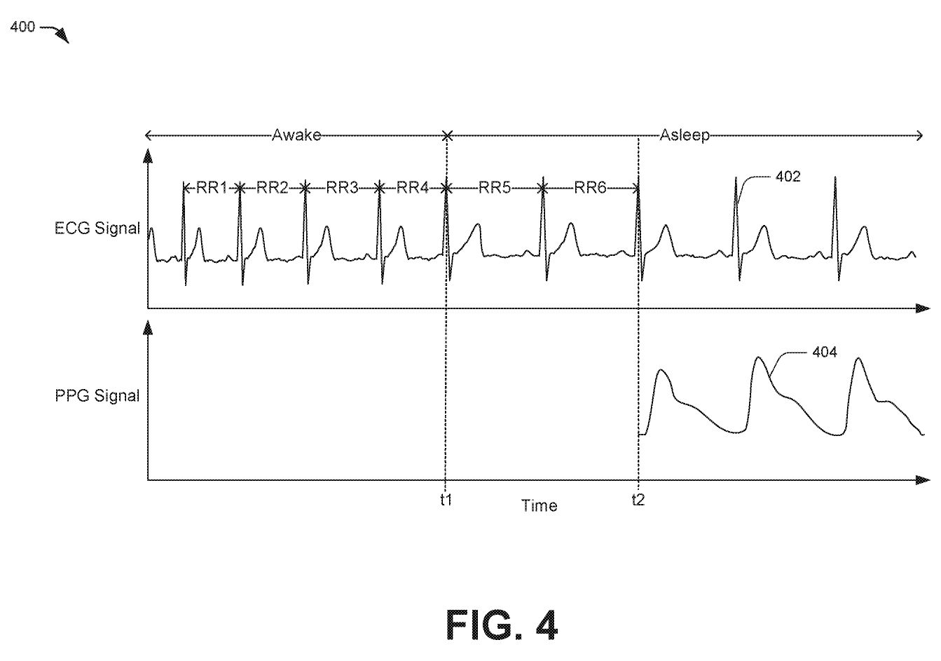

FIG. 4 illustrates a first example 400 of physiological measurement initiation using multiple sensors based on an ECG signal during awake and asleep states. Where appropriate, reference will be made to components previously introduced in FIGS. 1-3B.

The first example 400 includes an ECG signal 402 and a PPG signal 404 shown over time. By way of example, the ECG signal 402 may be acquired via the one or more sensors 202 of the monitoring device 104, and the PPG signal 404 may be acquired via the one or more sensors 118 of the auxiliary device 116, although variations are possible. Accordingly, although FIG. 4 will be described with respect to the monitoring device 104 acquiring the ECG signal 402 and the auxiliary device 116 acquiring the PPG signal 404, the techniques illustrated by the first example 400 may be applied in implementations where the monitoring device 104 acquires both the ECG signal 402 and the PPG signal 404, implementations where the auxiliary device 116 acquires the ECG signal 402 and the monitoring device 104 acquires the PPG signal 404, and so forth.

The ECG signal 402 includes successive R-R intervals labeled RR1, RR2, RR3, RR4, RR5, and RR6. An R-R interval, for instance, denotes a duration between successive R-wave peaks in the ECG signal 402, which corresponds to a duration between consecutive heartbeats. In the first example 400, the R-R intervals decrease in duration from RR1 through RR4, and the R-R intervals RR5 and RR6 have approximately equivalent durations, indicating stabilization of a reduced heart rate that may be indicative of sleep.

The first example 400 demonstrates use of ECG-based sleep detection to trigger selective sensor activation of a PPG sensor configured to acquire the PPG signal 404. In the first example 400, the person 102 falls asleep at a first time point t1, as shown by a transition from an “awake” period to an “asleep” period. The activation algorithm 120 analyzes the ECG signal 402 in real-time and evaluates whether the ECG signal 402 indicates predetermined criteria for the physiological condition 304 being present, such as the stabilization of the reduced heart rate. Accordingly, at a second time point t2, the activation algorithm 120 determines the physiological condition 304 is present and generates the activation signal 306 in response thereto. In the first example 400, the delay between the first time point t1 and the second time point t2 reflects an evaluation period where the activation algorithm 120 determines, based on the ECG signal 402 obtained during the “awake” period and the “asleep” period, that the person 102 is asleep, e.g., based on the reduced heart rate during sleep onset, which stabilizes in the first example 400 when the person 102 falls asleep. It is to be appreciated that the particular morphology and patterns of the ECG signal 402 shown in FIG. 4 are illustrative and non-limiting. Moreover, in at least one variation, the activation algorithm 120 extracts a respiration rate from the ECG signal 402 in addition to or as an alternative to evaluating the ECG signal 402 itself. Moreover, the ECG signal 402 may be combined with data measured by other sensors, such as accelerometer data as a non-limiting example.

In various implementations, the delay between the first time point t1 and the second time point t2 may vary based on a configuration of the activation algorithm 120. By way of example, the delay may be calibrated for a number of consecutive heartbeats, smoothing parameters, and/or hysteresis thresholds. Such calibration may enable the activation algorithm 120 to balance sensitivity to sleep onset against robustness to transient heart rate changes that may occur during transitions between wakefulness and sleep. By evaluating the stabilization of R-R intervals over multiple heartbeats, for instance, the activation algorithm 120 may avoid premature activation during brief periods of reduced heart rate that do not represent sustained sleep.

In one or more implementations, the monitoring device 104 continuously collects the ECG signal 402, and the analysis platform 106 executes the activation algorithm 120 to detect and confirm the physiological condition 304. Prior to the second time point t2, the PPG signal 404 is not acquired due to the one or more sensors 118 being deactivated (e.g., powered off). In response to the activation algorithm 120 detecting and confirming the physiological condition 304 at the second time point t2, the activation algorithm 120 transmits the activation signal 306 to the auxiliary device 116. Responsive to receiving the activation signal 306 at the second time point t2, the auxiliary device 116 activates the one or more sensors 118 to measure the PPG signal 404, which exhibits periodic waveforms corresponding to blood volume changes associated with cardiac cycles.

By activating collection of the PPG signal 404 after the activation algorithm 120 determines the physiological condition 304 is present at the second time point t2, the first example 400 illustrates selective sensor activation. In one or more implementations, the first example 400 may be applied in combined ambulatory cardiac monitoring and home sleep testing, where ECG data is collected continuously for cardiac assessment while PPG data is collected selectively during sleep periods for sleep analysis. The continuous collection of ECG data enables both cardiac arrhythmia detection throughout the monitoring period and sleep state detection for triggering PPG collection, thereby supporting dual clinical purposes with optimized resource utilization. The selective activation shown in the first example 400 reduces power consumption by maintaining a PPG sensor in an inactive state during periods when PPG data collection is not relevant to a clinical application.

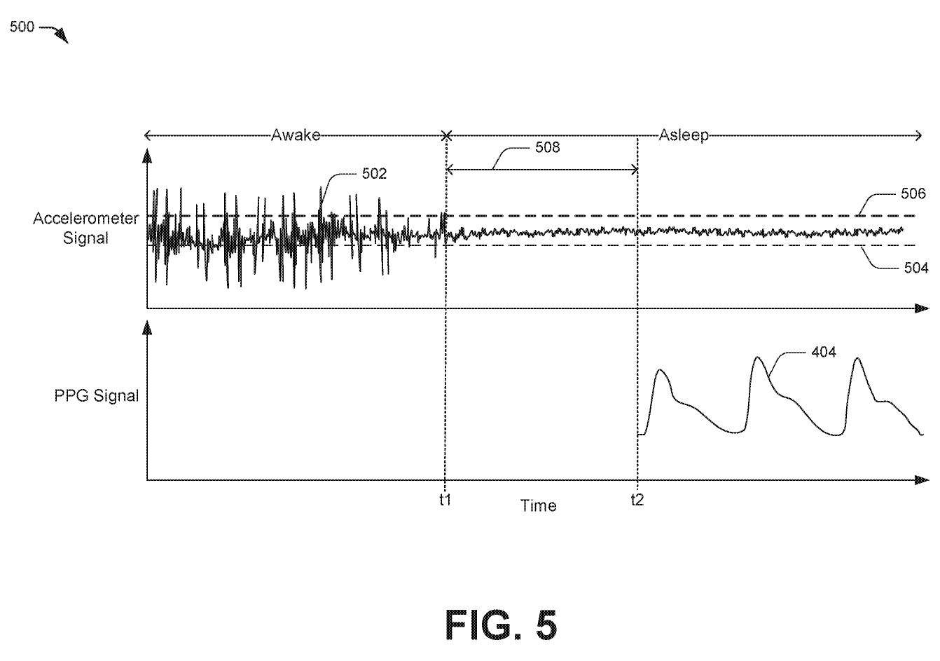

FIG. 5 depicts a second example 500 illustrating physiological measurement initiation using multiple sensors based on an accelerometer signal during awake and asleep states. Where appropriate, reference will be made to components previously introduced in FIGS. 1-4.

The second example 500 includes an accelerometer signal 502 and the PPG signal 404 shown over time. By way of example, the accelerometer signal 502 may be acquired via the one or more sensors 202 of the monitoring device 104, and the PPG signal 404 may be acquired via the one or more sensors 118 of the auxiliary device 116, although variations are possible. Accordingly, although FIG. 5 will be described with respect to the monitoring device 104 acquiring the accelerometer signal 502 and the auxiliary device 116 acquiring the PPG signal 404, the techniques illustrated by the second example 500 may be applied in implementations where the monitoring device 104 acquires both the accelerometer signal 502 and the PPG signal 404, implementations where the auxiliary device 116 acquires the accelerometer signal 502 and the monitoring device 104 acquires the PPG signal 404, and so forth.

The accelerometer signal 502 exhibits high-amplitude fluctuations during an awake period, indicating movement of the person 102. During a subsequent asleep period, the accelerometer signal 502 shows reduced amplitude and stabilized levels consistent with decreased movement. The second example 500 includes a lower threshold 504 and an upper threshold 506 that define bounds for a threshold range for differentiating activity from sleep. By way of example, the accelerometer signal 502 may indicate movement when the accelerometer signal 502 goes outside of the threshold range.

The second example 500 demonstrates use of accelerometer-based sleep detection to trigger selective sensor activation of a PPG sensor configured to acquire the PPG signal 404. In the second example 500, the person 102 falls asleep at a first time point t1, as shown by a transition from an “awake” period to an “asleep” period. The activation algorithm 120 analyzes the accelerometer signal 502 in real-time and evaluates whether the accelerometer signal 502 remains within the threshold range defined by the lower threshold 504 and the upper threshold 506 for a threshold duration 508. When the accelerometer signal 502 remains within the lower threshold 504 and the upper threshold 506 for the threshold duration 508, the activation algorithm 120 determines the physiological condition 304 is present. Accordingly, at a second time point t2, the activation algorithm 120 generates the activation signal 306 in response thereto. It is to be appreciated that the particular morphology and patterns of the accelerometer signal 502 shown in FIG. 5 are illustrative and non-limiting. Moreover, the accelerometer signal 502 may be combined with data measured by other sensors, such as ECG data as a non-limiting example.

In various implementations, the lower threshold 504, the upper threshold 506, and/or the threshold duration 508 may be configured based on a clinical application, such as to balance responsiveness of sensor activation against robustness to transient movement during sleep transitions, normal sleep movements such as position changes or periodic limb movements, and/or transient stillness during rest that is not sleep. A shorter threshold duration 508, for instance, may enable faster activation of the PPG sensor upon sleep onset, while a longer threshold duration 508 may reduce unwanted activations caused by brief periods of stillness during wakefulness. The lower threshold 504 and the upper threshold 506 may similarly be configured to define an acceptable range of movement that distinguishes sleep from wakefulness.

In one or more implementations, the monitoring device 104 continuously collects the accelerometer signal 502, and the analysis platform 106 executes the activation algorithm 120 to detect and confirm the physiological condition 304. Prior to the second time point t2, the PPG signal 404 is not acquired due to the one or more sensors 118 being deactivated (e.g., powered off). In response to the activation algorithm 120 detecting and confirming the physiological condition 304 at the second time point t2, the activation algorithm 120 transmits the activation signal 306 to the auxiliary device 116. Responsive to receiving the activation signal 306 at the second time point t2, the auxiliary device 116 activates the one or more sensors 118 to measure the PPG signal 404, which exhibits periodic waveforms corresponding to blood volume changes associated with cardiac cycles.

By activating collection of the PPG signal 404 after the activation algorithm 120 determines the physiological condition 304 is present at the second time point t2, the second example 500 illustrates selective sensor activation. The second example 500 demonstrates use of accelerometer-based sleep detection as an addition to or as an alternative to the ECG-based detection shown in the first example 400. In one or more implementations, the activation algorithm 120 may combine the analysis of both the ECG signal 402 data and the accelerometer signal 502 to improve an accuracy of detecting the physiological condition 304 and generating the activation signal 306. For example, the activation algorithm 120 may evaluate whether both cardiac interval stabilization and reduced movement are present before confirming the physiological condition 304, thereby increasing a confidence in sleep state detection and reducing unwanted activations.

The following discussion describes techniques that are implementable utilizing the previously described systems and devices. Aspects of the procedure (e.g., method) can be implemented in hardware, firmware, software, or a combination thereof. The procedure is shown as a set of blocks that specify operations that can be performed by one or more devices and are not necessarily limited to the orders shown for performing the operations by the respective blocks. One or more blocks of the procedure, for instance, specify operations that can be programmable by hardware (e.g., a processor, microprocessor, controller, or firmware) as executable instructions, thereby creating a special purpose machine for carrying out an algorithm (e.g., the activation algorithm 120 of FIG. 1) as illustrated by the flow diagram. As a result, the instructions are storable on a computer-readable storage medium that causes the hardware to perform the algorithm. In portions of the following discussion, reference will be made to FIGS. 1-5.

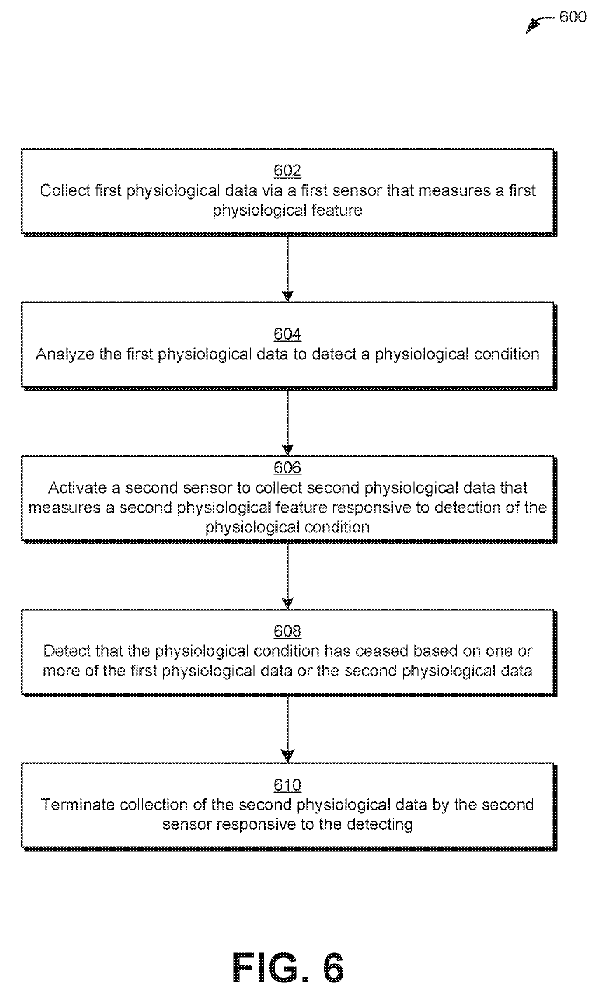

FIG. 6 illustrates a method 600 for physiological measurement initiation using multiple sensors. The method 600 may be implemented by the analysis platform 106, the monitoring device 104, the auxiliary device 116, and/or other suitable components of a physiological monitoring system. In at least one implementation, the method 600 is executed at least partially by a processing device, which may include or have access to the analysis platform 106 and the activation algorithm 120. In various implementations, the activation algorithm 120 may perform one or more operations of the method 600.

First physiological data is collected via a first sensor measuring a first physiological feature (block 602). By way of example, the first sensor may be implemented by the monitoring device 104 attached to a chest region of the person 102. The first physiological data may include one or more of electrocardiography (ECG) data or accelerometer data. The first physiological data may be collected continuously and buffered for real-time analysis. The continuous collection enables ongoing evaluation of a physiological state of the person 102 without relying on manual initiation, for example. The first physiological feature may be a cardiac feature determined or derived from the ECG data and/or a movement feature determined or derived from the accelerometer data.

The first physiological data are analyzed in real-time to detect a physiological condition (block 604). By way of example, the analysis may be performed by the analysis platform 106, such as via the activation algorithm 120. Additionally, or alternatively, the analysis may use one or more machine learning models configured to detect one or more physiological conditions based on various physiological data. In an example, detecting the physiological condition 304 includes a determination that, based on the analysis of the first physiological data 302, the person 102 is asleep. In some examples, the analysis is based on multiple sensor readings, e.g., a combination of ECG and accelerometer readings.

The analysis may include threshold evaluation, stabilization assessment over time, and/or other configurable detection criteria. For ECG-based detection, for example, the analysis may include evaluating R-R interval patterns from the ECG signal 402 over multiple heartbeats to determine whether cardiac intervals have stabilized at levels consistent with sleep. The number of consecutive heartbeats evaluated may be configured to balance sensitivity and specificity of sleep detection. For accelerometer-based detection, for example, analysis may include evaluating whether signal amplitude remains within the lower threshold 504 and the upper threshold 506 for the threshold duration 508. In yet another example, the analysis includes extracting a respiration rate from the ECG signal 402 and detecting the physiological condition 304 based on a lower respiration rate indicative of sleep.

A second sensor is activated to collect second physiological data measuring a second physiological feature in response to detection of the physiological condition (block 606). By way of example, the second sensor may be implemented by the auxiliary device 116 attached to an extremity of the person 102. The second physiological data 308 may include photoplethysmogram (PPG) data, for instance, and the second physiological feature may include SpO2, pulse rate, respiration rate, and/or another parameter determined or derived from PPG data. Activating the second sensor may include powering on the second sensor from a low-power or unpowered state. The activation may occur when detection criteria are satisfied, such as when the ECG signal 402 exhibits stabilization over multiple consecutive heartbeats and/or when the accelerometer signal 502 exhibits low movement indicative of sleep. In various examples, activation of the second sensor includes configuring the second sensor between an “on” state and an “off” state, e.g., to collect a reduced percentage of readings, such that various intermediate states of the second sensor are configurable.

In some cases, the first sensor and the second sensor may both be included in the monitoring device 104 or the auxiliary device 116. This configuration may allow for a more compact form factor while providing the ability to selectively activate sensors based on detected conditions.

The method 600 may be used to save storage space and battery life by selectively recording the second physiological data 308 when relevant. By selectively activating the second sensor, the amount of data collected and stored may be reduced, which may extend the monitoring period and/or reduce power consumption.

Cessation of the physiological condition is detected based on one or more of the first physiological data or the second physiological data (block 608). By way of example, the activation algorithm 120 may evaluate whether the first physiological feature measured in the first physiological data 302 no longer exhibits sleep-associated patterns and/or features, indicating a return to wakefulness. Alternatively, or in addition, the activation algorithm 120 may analyze the second physiological data 308 to detect patterns and/or features indicative of cessation of the physiological condition 304, such as changes in PPG waveform characteristics, pulse rate patterns, and/or oxygen saturation levels that indicate a transition from sleep to wakefulness. This ongoing analysis allows the method 600 to dynamically respond to changes in the physiological state of the person 102.

In at least one variation, however, the cessation may be determined based on a predetermined time duration following activation of the second sensor, a predetermined number of measurements or data samples collected by the second sensor, or satisfaction of other predetermined criteria such as data quality metrics, storage capacity thresholds, and/or battery level thresholds.

Collection of the second physiological data by the second sensor is terminated responsive to the detecting (block 610). By way of example, the activation algorithm 120 may generate the deactivation signal 310, which is transmitted to the auxiliary device 116 to deactivate the second sensor (e.g., the one or more sensors 118). By terminating the second sensor when the physiological condition 304 is no longer present and/or when the predetermined criteria are satisfied, the method 600 further optimizes power consumption and data storage. Termination may include returning the second sensor to a low-power state or deactivating data collection while maintaining the sensor in a standby mode for rapid reactivation if the physiological condition 304 is detected again.

Alternatively, the auxiliary device 116 may automatically deactivate the second sensor upon expiration of the predetermined time duration, completion of the predetermined number of measurements, and/or satisfaction of the other predetermined criteria.

Accordingly, in one example, the method 600 may include determining a first condition (e.g., the physiological condition 304) is present at a first time, and in response thereto, activating the second sensor to collect the second physiological data 308; and determining a second condition (which occurs when the first condition is not present, e.g., when the physiological condition 304 is not present) at a second time, and in response thereto, deactivating the second sensor to cease collecting the second physiological data 308. In some examples, activating the second sensor to collect the second physiological data 308 occurs while or during the first condition, and deactivating the second sensor to cease collecting the second physiological data 308 occurs while the first condition is not present and/or while or during the second condition. Further, the first condition may include the individual being asleep, and the second condition may include the individual being awake.

Further, instructions stored in memory may include instructions for determining the first condition from a signal received from the first sensor (e.g., the one or more sensors 202), and in response, activating the second sensor by instructions for sending an activation signal 306 to the second sensor (e.g., the one or more sensors 118) to power on the second sensor and begin data collection. Instructions stored in memory may further define a configuration in which to activate the second sensor (e.g., a sampling current, a drive current, and the like). Consider an example where the first condition includes the user being asleep, but an ambient light level is high. In such an example, the activation signal 306 to the second sensor may instruct the second sensor to turn on with high current and/or high ambient noise cancellation/filtering. Alternatively, or in addition, the sampling rate for the second sensor may be defined by (e.g., proportional to) the level of the accelerometer activity measured by the first sensor, where more activity results in a higher sampling rate.

Instructions stored in memory may further include instructions for determining the second condition from the signal received from the first sensor and/or from a signal received from the second sensor, and in response, deactivating the second sensor by instructions for sending the deactivation signal 310 to the second sensor to power off the second sensor and/or cease data collection. As a further example, instructions stored in memory may include instructions for differentiating between the first condition and the second condition based on the signal received from the first sensor and/or the second sensor and determining whether to perform one or more of activating the second sensor and deactivating the second sensor based on a determination of whether the first condition is present and a determination of whether the second condition is present.

In this way, the method 600 provides a dynamic approach to physiological monitoring that enables efficient use of multiple sensors based on a real-time analysis of physiological data. By selectively activating and deactivating sensors based on detecting a physiological condition (or lack thereof), the method 600 optimizes power consumption and data storage while maintaining comprehensive monitoring capabilities. This approach may be particularly useful for applications such as combined ambulatory cardiac monitoring and home sleep testing, where a continuous collection of certain physiological data (e.g., ECG data) enables both an ongoing cardiac assessment and a detection of physiological condition(s) that trigger selective collection of additional physiological data (e.g., PPG data) when relevant to a clinical application.

### Machine Learning and AI in Physiological Measurement Initiation Using Multiple Sensors

The previous examples describe various instances of artificial intelligence (“AI”) models or machine learning models, such as with respect to the activation algorithm 120 or the prediction system 114. In one or more examples, an AI model, e.g., a machine learning model, refers to a computer representation that is tunable (e.g., through training and retraining) based on inputs to approximate unknown functions, automatically and without user intervention, without being actively programmed by a user. For instance, the term machine learning model includes a model that utilizes algorithms to learn from, and make predictions on, known data by analyzing training data to learn and relearn to generate outputs that reflect patterns and attributes of the training data.

In the context of physiological measurement initiation using multiple sensors, machine learning models are implementable (e.g., by one or more processing devices of the analysis platform 106) to analyze physiological data patterns such as to identify physiological conditions, determine when to activate or deactivate sensors, and generate activation or deactivation signals. For example, the activation algorithm 120 or the prediction system 114 may each utilize one or more machine learning models to process physiological data such as ECG signals, accelerometer signals, PPG signals, heart rate variability, respiratory patterns, and/or other measurements collected by the monitoring device 104 and/or the auxiliary device 116. Examples of machine learning models applicable to physiological condition detection and sensor activation include neural networks, convolutional neural networks (CNNs) such as for analyzing waveform data and identifying patterns and/or features indicative of sleep or other physiological states, long short-term memory (LSTM) neural networks such as to analyze temporal physiological patterns and detect transitions between physiological states, generative adversarial networks (GANs), decision trees (e.g., for classification of physiological conditions), support vector machines, linear regression, logistic regression for binary state detection, Bayesian networks, random forest learning for feature importance in physiological condition detection, dimensionality reduction algorithms, boosting algorithms, deep learning neural networks, and so forth.

A machine learning model, for instance, is configurable using a plurality of layers having, respectively, a plurality of nodes. The plurality of layers is configurable to include an input layer, an output layer, and one or more hidden layers. In the context of physiological measurement initiation, the input layer may receive various physiological parameters from the measurements 108, such as ECG features, R-R intervals, accelerometer data, heart rate patterns, respiratory signals, motion data, and timing information from multiple devices. The hidden layers, for instance, process these inputs through weighted connections to identify complex patterns indicative of physiological conditions such as sleep states, e.g., patterns that are not detectable using conventional threshold-based methods. The output layer may produce physiological condition classifications, activation/deactivation signal triggers, or generate the one or more predictions 110 that incorporate selectively collected physiological data from multiple sensors. Calculations are performed by the nodes within the layers via hidden states through a system of weighted connections that are “learned” during training of the machine learning model to implement a variety of physiological condition detection and sensor activation tasks.

In order to train the machine learning model for physiological measurement initiation, training data are received that provide examples of “what is to be learned” by the machine learning model, i.e., as a basis to learn patterns and/or features from the data. For sensor activation applications, the training data may include labeled datasets of physiological measurements with known physiological states, such as ECG and accelerometer data recorded during verified sleep and wake periods, respiratory signals with confirmed sleep stage annotations, heart rate variability patterns associated with different activity levels, and/or physiological data with labeled transitions between physiological states. A machine learning system that includes the machine learning model, for instance, collects and preprocesses the training data that include input features (e.g., ECG waveforms, R-R intervals, accelerometer signals, respiratory patterns, heart rate intervals) and corresponding target labels (e.g., “asleep,” “awake,” “transition to sleep,” or specific physiological state classifications).

The machine learning system is further operable to initialize various parameters of the machine learning model, which are usable by the machine learning model as internal variables to represent and process information during training. These parameters are further usable to represent inferences gained through training. In one or more implementations, the training data are separated into batches to improve processing and optimization efficiency of the parameters of the machine learning model during training, which may be beneficial for model accuracy when processing large volumes of physiological time-series data from multiple devices with varying sampling rates and temporal characteristics.

The training data are then received by the machine learning model as inputs and used to generate predictions based on a current state of parameters of layers and corresponding nodes of the model, a result of which is output as output data, e.g., a physiological condition classification, activation signal trigger, state transition probability, or the like. For example, the analysis platform 106 includes a machine learning model that is trained to recognize patterns and/or features in physiological data that correlate with sleep states or other physiological conditions, which enables the activation algorithm 120 to generate accurate activation signals and the prediction system 114 to process selectively collected physiological data for improved diagnostic accuracy.

Training of the machine learning model can include calculation of a loss function to quantify a loss associated with operations performed by nodes of the machine learning model. The loss function is configurable in various ways to control operation or functionality of the machine learning model. For instance, the loss function may be designed to prioritize accuracy in physiological condition detection while minimizing false activations that could lead to unwarranted power consumption or missed data collection opportunities. Calculation of the loss function, for instance, includes comparing a difference between predictions specified in the output data (e.g., predicted physiological states or activation decisions) with target labels specified by the training data (e.g., verified ground truth physiological states). The loss function is configurable in a variety of ways, examples of which include regret, quadratic loss function as part of a least squares technique for continuous state probability parameters, cross-entropy loss for physiological state classification tasks, custom loss functions that incorporate power efficiency requirements or data collection priorities specific to particular physiological monitoring applications, and so forth.

The training data are usable to support a variety of usage scenarios in physiological measurement initiation. For example, the machine learning model can be trained to detect specific patterns in physiological data (e.g., ECG and accelerometer data) that enable accurate sleep state detection, identify respiratory patterns indicative of sleep onset, recognize motion patterns that distinguish sleep from wakefulness, or detect subtle physiological changes that may improve activation timing precision. The models can be configured to operate within computational constraints of real-time condition detection while providing accurate activation decisions. The models can further be reconfigured, e.g., with expanded capabilities, for more sophisticated physiological state analysis when processing historical data or performing detailed diagnostic assessments. This adaptive approach enables efficient use of computational resources devoted to machine learning processes while ensuring comprehensive physiological condition detection capabilities are available when needed for accurate selective sensor activation across multiple devices and measurement modalities.

It should be understood that many variations are possible based on the disclosure herein. Although features and elements are described above in particular combinations, each feature or element is usable alone without the other features and elements or in various combinations with or without other features and elements.

What is claimed is:

1. A method for physiological measurement initiation, comprising:
collecting first physiological data from a first sensor measuring a first physiological feature of an individual;
analyzing the first physiological data in real-time to detect a physiological condition of the individual; and
responsive to detecting the physiological condition, activating a second sensor to collect second physiological data measuring a second physiological feature of the individual.

2. The method of claim 1, further comprising:
detecting that the physiological condition has ceased based on one or more of the first physiological data or the second physiological data; and
terminating collection of the second physiological data by the second sensor responsive to the detecting.

3. The method of claim 2, wherein terminating collection of the second physiological data by the second sensor responsive to the detecting comprises transmitting a deactivation signal to the second sensor responsive to the detecting.

4. The method of claim 1, wherein the activating the second sensor includes powering on the second sensor.

5. The method of claim 1, wherein the first sensor is included in a wearable monitoring device attached to a chest region of the individual, and the second sensor is included in an auxiliary device attached to an extremity of the individual.

6. The method of claim 1, wherein the first sensor and the second sensor are included in a single wearable monitoring device.

7. The method of claim 1, wherein the first physiological data include one or more of electrocardiography (ECG) data or accelerometer data, and the second physiological data include photoplethysmogram (PPG) data.

8. The method of claim 1, wherein the physiological condition is an asleep condition of the individual.

9. The method of claim 1, wherein the first physiological data comprise ECG data, and analyzing the first physiological data to detect the physiological condition of the individual comprises:
detecting, as the first physiological feature, a cardiac feature in the ECG data; and
detecting the physiological condition based on the cardiac feature.

10. The method of claim 1, wherein the first physiological data comprise accelerometer data, and analyzing the first physiological data to detect the physiological condition of the individual comprises:
detecting, as the first physiological feature, a movement feature in the accelerometer data; and
detecting the physiological condition based on the movement feature.

11. A system for physiological measurement initiation, comprising:
a first sensor configured to collect first physiological data measuring a first physiological feature of an individual;
a second sensor configured to collect second physiological data measuring a second physiological feature of the individual; and
a processing device configured to:
analyze the first physiological data in real-time to detect a physiological condition of the individual; and
activate the second sensor to collect the second physiological data responsive to detecting the physiological condition.

12. The system of claim 11, wherein the processing device is further configured to:
detect that the physiological condition has ceased based on one or more of the first physiological data or the second physiological data; and
in response to detecting that the physiological condition has ceased, terminate collection of the second physiological data by the second sensor.

13. The system of claim 11, wherein the processing device is further configured to:
terminate collection of the second physiological data by transmitting a deactivation signal to the second sensor after a predetermined time duration or after a predetermined number of measurements has been collected by the second sensor.

14. The system of claim 11, wherein the processing device is configured to activate the second sensor by transmitting an activation signal to the second sensor.

15. The system of claim 11, wherein the first sensor is included in a wearable monitoring device configured to be attached to a chest region of the individual, and the second sensor is included in an auxiliary device configured to be attached to an extremity of the individual.

16. The system of claim 11, wherein the first sensor and the second sensor are included in a single wearable monitoring device.

17. The system of claim 11, wherein the first sensor is one or more of an electrocardiography (ECG) sensor or an accelerometer sensor, and the second sensor is a photoplethysmogram (PPG) sensor.

18. A method for physiological measurement initiation, comprising:
continuously collecting first physiological data from a first sensor that measures a first physiological feature of an individual over an observation period;
analyzing the first physiological data in real-time to detect a physiological condition of the individual at a first time point during the observation period;
responsive to detecting the physiological condition, activating a second sensor at the first time point to collect second physiological data measuring a second physiological feature of the individual;
at a second time point during the observation period, detecting cessation of the physiological condition based on one or more of the first physiological data or the second physiological data; and
deactivating the second sensor at the second time point responsive to detecting the cessation of the physiological condition.

19. The method of claim 18, wherein the first physiological data comprise one or more of ECG data or accelerometer data, the second physiological data comprise PPG data, and the physiological condition is an asleep condition of the individual.

20. The method of claim 18, wherein the first sensor is included in a wearable monitoring device attached to a chest region of the individual, and the second sensor is included in an auxiliary device attached to an extremity of the individual.
